(19) United States
(12) Patent Application Publication
(10) Pub. No.: US 2014/0296600 A1
(43) Pub. Date: Oct. 2, 2014

(54) PRODUCTION OF PARA-XYLENE BY CATALYTICALLY REACTING 2,5-DIMETHYLFURAN AND ETHYLENE IN A SOLVENT

(71) Applicants: University of Delaware, Newark, DE (US); University of Massachusetts, Boston, MA (US)

(72) Inventors: Paul Jakob Dauenhauer, Sunderland, MA (US); Christopher Luke Williams, Amherst, MA (US); Dionisios G. Vlachos, Voorhees, NJ (US); Raul F. Lobo, Newark, DE (US); Chun-Chih Chang, Amherst, MA (US); Wei Fan, Amherst, MA (US)

(73) Assignees: University of Delaware, Newark, DE (US); University of Massachusetts, Boston, MA (US)

(21) Appl. No.: 14/230,903

(22) Filed: Mar. 31, 2014

Related U.S. Application Data
(60) Provisional application No. 61/807,099, filed on Apr. 1, 2013.

(51) Int. Cl. C07C 2/86

(52) U.S. Cl. CPC C07C 2/865
USPC 585/469

(57) ABSTRACT

The present invention provides a renewable route to para-xylene via cycloaddition of ethylene and 2,5-dimethylfuran and subsequent dehydration with high selectivity and high yields using acidic heterogeneous catalysts and a solvent for 2,5-dimethylfuran. The use of a solvent shows significant effects in the reduction of competing side reactions including hydrolysis of 2,5-dimethylfuran to 2,5-hexanedione, alkylation of p-xylene, and polymerization of 2,5-hexanedione.

### RELATED APPLICATION

This application claims the benefit of U.S. Provisional Application No. 61/807,099, filed on Apr. 1, 2013. The entire teachings of that application are incorporated herein by reference.

### GOVERNMENT SUPPORT

This invention was made with government support under the Catalysis Center for Energy Innovation, an Energy Frontier Research Center funded by the U.S. Department of Energy, Office of Science, Office of Basic Energy Sciences under Award N. DE-SC0001004. The government has certain rights in the invention.

### BACKGROUND OF THE INVENTION

The need for sustainable production of everyday materials in addition to market volatility of petroleum-based feedstocks has motivated research into the production of renewable aromatic chemicals from biomass.^{[1-6]} Cellulose and hemicellulose comprise a significant fraction of lignocellulosic biomass and provide six-carbon (e.g., glucose) and five-carbon (e.g., xylose) sugars as primary feedstocks for production of renewable aromatic chemicals with six-carbon aromatic ring structures.^{[7]} Specific chemicals of interest include para-xylene, a feedstock for polyethylene terephthalate (PET), and toluene, an important monomer for polyurethane products.^{[8-10]}

Recent advances have demonstrated the potential of using cycloaddition as a method for producing renewable aromatic chemicals.^{[11-12]} As an example, it is possible to synthesize para-xylene from 2,5-dimethylfuran and ethylene in the presence of a catalyst.^{[12]} However, known methods for producing para-xylene provide low selectivity and/or low yields.

Therefore, there remains a need for the development of improved methods and processes for the production of para-xylene.

### SUMMARY OF THE INVENTION

The invention is the use of solvents in the catalytic cycloaddition reaction of 2,5-dimethylfuran with ethylene.

In one embodiment, the method for producing para-xylene comprises reacting 2,5-dimethylfuran (“DMF”) with ethylene under cycloaddition reaction conditions in the presence of an acidic heterogeneous catalyst in a solvent for 2,5-dimethylfuran to produce para-xylene (“p-xylene”), wherein the solvent is present in an amount of at least 20 wt %.

In another embodiment, the method for producing para-xylene comprises reacting 2,5-dimethylfuran with ethylene under cycloaddition reaction conditions in the presence of an acidic zeolite beta (“H-beta”) catalyst and a solvent for 2,5-dimethylfuran to produce para-xylene.

In yet another embodiment, the method for producing para-xylene comprises reacting 2,5-dimethylfuran with ethylene under cycloaddition reaction conditions in the presence of an acidic zeolite Y (“H—Y”) catalyst and a solvent for 2,5-dimethylfuran to produce para-xylene, wherein the solvent is present in an amount of at least 20 wt %.

The methods of the current invention are carried out at an elevated temperature, preferably at least 100° C., and an elevated pressure, preferably at least 5 bar (73 psi).

The acidic heterogeneous catalysts of the present invention can comprise a variety of catalysts including, but not limited to, a zeolite molecular sieve, activated carbon, silica, alumina and a non-zeolitic molecular sieve. Several solvents types are suitable for the methods of the current invention including various polar and nonpolar solvents.

It has been found that this invention produces para-xylene in high yields and high selectivity. For example, the methods described herein can be used to obtain para-xylene in >90% yield and >80% selectivity.

### BRIEF DESCRIPTION OF THE DRAWINGS

The foregoing will be apparent from the following more particular description of example embodiments of the invention, and as illustrated in the accompanying drawings.

FIG. 1 is a bar graph illustrating the effects of solvent on the conversion of 2,5-dimethylfuran and the selectivity for the formation of para-xylene with H-beta zeolite catalyst. (See Example 1). Both the experiments with solvent and the experiments without solvent were run three times each. FIG. 1 shows the combined data of the three experiments run under both of the conditions (with and without solvent) provided in Example 1. The error bars for the combined data were calculated in a 90% confidence interval.

FIG. 2 is a schematic illustrating the pathway for conversion of 2,5-dimethylfuran to para-xylene and for the formation of undesired side products from competing side reactions.

FIG. 3 is a graph illustrating the concentration of side products as a function of 2,5-dimethylfuran conversion for the reaction of 2,5-dimethylfuran in heptane with ethylene at 250° C. in the presence of H-beta zeolite catalyst (Si/Al=12.5). (See Example 2). FIG. 3 shows the combined data of three experiments run under the conditions provided in Example 2. The error bars for the combined data were calculated in a 90% confidence interval.

FIG. 4 is a graph illustrating the selectivity of para-xylene and concentration of 2,5-hexanedione as a function of 2,5-dimethylfuran conversion for the reaction of 2,5-dimethylfuran in heptane with ethylene at 250° C. in the presence of H-beta zeolite catalyst (Si/Al=12.5). (See Example 2). FIG. 4 shows the combined data of three experiments run under the conditions provided in Example 2. The error bars for the combined data were calculated in a 90% confidence interval.

FIG. 5 is a bar graph illustrating the effects on reaction rates observed upon an increase in reaction temperature from 200° C. to 300° C. when the cycloaddition reaction is carried out in the presence of an H—Y zeolite catalyst (Si/Al=30), ethylene at 62 bar (900 psig) and pure 2,5-dimethylfuran. FIG. 5 also illustrates the further increases in yield and selectivity of para-xylene observed as a result of the addition of a solvent (18.5 wt % 2,5-dimethylfuran/81.5 wt % n-heptane) at 300° C. (See Example 3).

FIG. 6 is a bar graph illustrating the effect on the selectivity for the formation of para-xylene observed upon an increase in reaction temperature from 200° C. to 300° C. when the cycloaddition reaction is carried out in the presence of a zeolite catalyst, ethylene at 14 bar (200 psig) and pure 2,5-dimethylfuran. FIG. 6 also illustrates the further increases in yield and selectivity of para-xylene observed as a result of the addition of a solvent, such that 2,5-dimethylfuran is initially at 18.5 wt %, and as a result of the substitution of an H—Y zeolite catalyst for an H-beta zeolite catalyst.

FIG. 7 is a graph illustrating the effect on the selectivity for the formation of para-xylene observed upon an increase of the initial concentration of 2,5-dimethylfuran from 0.3M to 1.7M when the cycloaddition reaction is carried out in the presence H-beta zeolite catalyst and 14 bar (200 psig) of ethylene at 200° C.

FIG. 8 is a bar graph illustrating the effect on initial reaction rates observed in the presence siliceous catalysts and catalysts containing a varying number of Brønsted acid sites when the cycloaddition reaction is carried out in the presence of ethylene at 57 bar (827 psig) and pure 2,5-dimethylfuran at 300° C. The error bars for the data were calculated in a 90% confidence interval.

FIG. 9 is a graph illustrating the effect on the rate limiting step observed upon an increase in the Brønsted acid site loading (by increasing catalyst loading) to an effective concentration greater than 2.5 mM. The reaction was carried out in the presence of various Brønsted acid catalysts, ethylene at 38 bar (550 psig) and 18.5 wt % 2,5-dimethylfuran in n-heptane at 250° C. At low catalyst loadings, the effective Brønsted acid site concentration is so low that the dehydration step of the cycloaddition reaction is rate limiting. However, at high catalyst loadings where the effective acid site concentration is above about 2.5 mM, the cycloaddition step is rate limiting.

FIG. 10A is a graph illustrating the measured activation energy observed when the dehydration limited reaction (where the dehydration step is rate limiting) is carried out in the presence of a low loading of H-beta zeolite catalyst such that the effective acid site concentration within the reaction vessel is about 1.25 mM. FIG. 10B is a graph illustrating the measured activation energy observed when the dehydration limited reaction is carried out in the presence of a low loading of H—Y zeolite catalyst such that the effective acid site concentration within the reaction vessel is 1.25 mM.

FIG. 10C is a graph illustrating the measured activation energy observed when the cycloaddition limited reaction (where the cycloaddition step is rate limiting) is carried out in the presence of an H-beta zeolite catalyst at an effective acid site concentration within the reaction vessel of about 3.25 mM. FIG. 10D is a graph illustrating the measured activation energy observed when the cycloaddition limited reaction is carried out in the presence of an H—Y zeolite catalyst at an effective acid site concentration within the reaction vessel of about 3.25 mM.

FIG. 11A is a graph illustrating the measured reaction order for 2,5-dimethylfuran observed when the cycloaddition limited reaction is carried out in the presence of an H—Y zeolite catalyst at an effective acid site concentration of about 3.25 mM, 2,5-dimethylfuran and ethylene at 14 bar (200 psig) at 200° C. 2,5-Dimethylfuran concentration was varied from about 0.3M to about 3.3 M. FIG. 11B is a graph illustrating the measured reaction order for ethylene observed when the cycloaddition limited reaction is carried out in the presence of an H—Y zeolite catalyst at an effective acid site concentration of about 3.25 mM, 18.5 wt % (1.4 M) 2,5-dimethylfuran, and increasing concentrations of ethylene from about 0.04M to about 0.18M at 200° C. Ethylene concentration was increased by pressurizing the reaction vessel from about 7 bar to about 34 bar (100 psig to about 500 psig) at 200° C.

FIG. 12A is a graph illustrating the effect on para-xylene isomerization observed in the absence of 2,5-dimethylfuran and ethylene when the reaction is carried out in the presence of 0.45 g H—Y an zeolite catalyst (Si/Al=2.6) with an effective acid site concentration of about 1.25 mM, nitrogen at 55 bar (800 psig), 1M para-xylene in heptane and tridecane as an internal standard at 300° C. FIG. 12B is a graph illustrating the effect on para-xylene isomerization observed in the absence of 2,5-dimethylfuran and ethylene when the reaction is carried out in the presence of 1.45 g of an H—Y zeolite catalyst (Si/Al=2.6) with an effective acid site concentration of about 1.25 mM, nitrogen at 55 bar (800 psig), 1M para-xylene in heptane and tridecane as an internal standard at 300° C. The increase in para-xylene isomerization indicates that the extent of isomerization is limited by catalyst deactivation rather than equilibrium of the para-xylene product.

FIGS. 13A and 13B illustrate the effects on para-xylene isomerization observed upon changes in the concentration of 2,5-dimethylfuran relative to the concentration of para-xylene in the presence of a zeolite catalyst, nitrogen at 55 bar (800 psig), heptane, and tridecane as an internal standard at 300° C. FIG. 13A is a graph illustrating the effect on para-xylene isomerization observed at a concentration ratio of para-xylene to 2,5-dimethylfuran of 5 to 1 and in the presence of an H—Y zeolite catalyst (Si/Al=2.6). FIG. 13B is a graph illustrating the effect on para-xylene isomerization observed at a concentration ratio of para-xylene to 2,5-dimethylfuran of 50 to 1 and in the presence of an H—Y zeolite catalyst (Si/Al=2.6). FIG. 13C is a graph illustrating the effect on para-xylene isomerization observed at a concentration ratio of para-xylene to 2,5-dimethylfuran of 5 to 1 and in the presence of an H-beta zeolite catalyst (Si/Al=12.5). FIG. 13D is a graph illustrating the effect on para-xylene isomerization observed at a concentration ratio of para-xylene to 2,5-dimethylfuran of 50 to 1 and in the presence of an H-beta zeolite catalyst (Si/Al=12.5).

FIGS. 14A and 14B illustrate the effects on para-xylene isomerization observed upon changes in the concentration of 2,5-hexanedione relative to the concentration of para-xylene in the presence of an H—Y zeolite catalyst (Si/Al=2.6), nitrogen at 55 bar (800 psig), heptane, and tridecane as an internal standard at 300° C.

FIG. 14A is a graph illustrating the effect on para-xylene isomerization observed at a concentration ratio of para-xylene to 2,5-hexanedione of 5 to 1. FIG. 14A shows that 2,5-dimethylfuran is formed by the cyclization and dehydration of the diketone. FIG. 14B is a graph illustrating the effect on para-xylene isomerization observed at a concentration ratio of para-xylene to 2,5-hexanedione of 50 to 1.

FIG. 15A is a graph illustrating the effect of ethylene (55 bar (800 psig)) on para-xylene isomerization observed in the presence of an H—Y zeolite catalyst (Si/Al=2.6), para-xylene, heptane, and tridecane as an internal standard at 300° C. FIG. 15B is a graph illustrating the effect of water on para-xylene isomerization observed in the presence of an H—Y zeolite catalyst (Si/Al=2.6), nitrogen at 55 bar (800 psig), para-xylene, heptane, and tridecane as an internal standard at 300° C.

FIG. 16A is a ²⁷Al-NMR spectrum of an H—Y zeolite catalyst (Si/Al=2.6) that was recovered after a cycloaddition reaction and that was not regenerated by coke burnoff (“spent”). FIG. 16B is a ²⁷Al-NMR spectrum of H—Y zeolite catalyst (Si/Al=2.6) that has not been used in a cycloaddition reaction (“fresh”).

FIG. 17i is a diffuse reflectance infrared fourier transform spectrum (DRIFTS) of degassed H—Y zeolite catalyst. FIG. 17ii is a DRIFT spectrum of para-xylene absorbed on an H—Y zeolite catalyst at 120° C. FIG. 17iii is an DRIFT spectrum of 1 to 50 mixture of 2,5-dimethylfuran to para-xylene absorbed on an H—Y zeolite catalyst. FIG. 17iv is a DRIFT spectrum showing the absence of Brønsted acid sites. FIGS. 17v and 17vi shows the DRIFT spectrum of 2,5-dimethylfuran absorbed on an H—Y zeolite catalyst after heating to 300° C. FIG. 17vii shows the DRIFT spectrum of para-xylene absorbed on an H—Y zeolite catalyst after heating to 300° C.

FIG. 18A is the thermogravimetry curve of absorbed 2,5-dimethylfuran on an H—Y zeolite catalyst. FIG. 18B is the thermogravimetry curve of absorbed 2,5-dimethylfuran on an H-beta zeolite catalyst. FIG. 18C is the thermogravimetry curve of absorbed para-xylene on an H—Y zeolite catalyst. FIG. 18D is the thermogravimetry curve of absorbed para-xylene on an H-beta zeolite catalyst.

### DETAILED DESCRIPTION OF THE INVENTION

The present invention is directed to an improved method for producing para-xylene (p-xylene). The method employs cycloaddition reaction conditions and includes the use of a solvent for the production of para-xylene in high yields and selectivities.

In one aspect, the invention provides a method for producing para-xylene comprising reacting 2,5-dimethylfuran with ethylene under cycloaddition reaction conditions in the presence of an acidic heterogeneous catalyst and a solvent to produce para-xylene. The term “catalyst,” as defined herein, refers to an agent that reduces the activation energy needed for the desired reaction increasing the rate of the reaction without itself undergoing any permanent chemical change. “Catalytic,” as used herein, means a reaction employing a catalyst.

As defined herein, “acidic heterogeneous catalyst” refers to a catalyst containing acidic sites whose phase (e.g., solid, liquid or gas) is different from the phase of the reactants. For example, the reactants (e.g., 2,5-dimethylfuran, heptanes) and product (e.g., para-xylene) can be in the liquid or gas phase while the catalyst is a solid. The acidic sites on the catalyst can be Lewis acid or Brønsted acid sites. The acidic heterogeneous catalysts for use with the current invention contain an acid site density of at least about 0.01 mmol/gram (g) of catalyst. As used herein, the “acid site density of mmol/g” means the number (in moles) of acid sites per unit mass of the acidic heterogeneous catalyst. Atomic Si/Al ratios are often used in the practice of catalysis to refer to the inverse of the acid site density or the number of non-acid sites (SiO₂) present per each acid site (HAlO₂). In one embodiment, the acidic heterogeneous catalyst contains a Brønsted acid site density of at least about 0.01 mmol/g. In another embodiment, the acidic heterogeneous catalyst contains a Lewis acid site density of at least about 0.01 mmol/g. Suitable acidic heterogeneous catalysts include, but are not limited to zeolite molecular sieves, activated carbon, silica, alumina and non-zeolitic molecular sieves.

Zeolite molecular sieves (“zeolites”) are crystalline aluminosilicate materials composed of corner-sharing AlO₄ and SiO₄ tetrahedra joined into three-dimensional frameworks having pores of molecular dimensions. The presence of aluminum in the zeolite framework results in a negative charge that is balanced by cations. The acidity of a zeolite is also affected by its composition where the acidity or Brønsted acid site concentration is equal to the concentration of aluminum present in the zeolite framework. As used herein, the terms “acidity”, “lower acidity” and “higher acidity” refer to the concentration of acid sites regardless of the strength of such acid sites.

A representative empirical formula of a zeolite molecular sieve is

M_{2/n}0.xAlO₃.ySiO₂.zH₂O

where M represents the exchangeable cation of valence n. M is generally a Group I or II ion (e.g., proton (H+), sodium (Na), magnesium (Mg), etc.), although other metal, non-metal and organic cations can also balance the negative charge created by the presence of aluminum (Al) in the structure. The symbols x, y and z are generally in the range of 1 to 300, more preferably 1 to 100, even more preferably 1 to 40. The framework may contain cages and channels of discrete size, which are normally occupied by water.

In addition to Si⁴⁺ and Al³⁺, other elements can also be present in the molecular sieve zeolitic framework. They need not be isoelectronic with Si⁴⁺ or Al³⁺, but must be able to occupy framework sites. For example, tin oxide germanium oxide, and mixtures thereof can replace the silica portion. Boron oxide, iron oxide, indium oxide, gallium oxide, and mixtures thereof can replace the alumina portion. Unless otherwise specified, the terms “zeolite” and “zeolite material” as used herein, shall mean any naturally occurring or synthetic crystalline substance with a structure characterized by a framework of linked tetrahedral, each consisting of four oxygen atoms surrounding a cation and include not only materials containing silicon and, optionally, aluminum atoms in the crystalline lattice structure thereof, but also materials which contain suitable replacement atoms for such silicon and aluminum. Aluminosilicate zeolites display a net negative framework charge, but other molecular sieve frameworks may be electrically neutral.

The zeolites for use with the invention include both natural and synthetic zeolites and comprise zeolites of various structural types. According to IUPAC rules, each zeolite structure is assigned a three-letter code that identifies the specific connectivity of the atoms in a given framework. This three letter code is used to identify unambiguously the structure of a zeolite material, independently of composition. One example is the zeolite Y which has a framework of the FAU-type. Examples of other zeolite structure types suitable for use in the invention include, but are not limited to, types such as BEA, CHA, EMT, ERI, EUO, FAU, FER, HEU, KFI, LEV, LTA, LTL, MAZ, MEI, MEL, MFI, MOR, MTT, MTW, and TON. Other framework-types as recognized by the structure-commission of the International Zeolite Association are included by extension. Synthetic zeolites are usually prepared by crystallization from a supersaturated synthesis mixture. The resulting crystalline product is then dried and calcined (e.g., heated to high temperature) to produce a zeolite powder.

The framework of a zeolite molecular sieve is based on an extensive three-dimensional network in which the polyhedral sites, usually tetrahedral, are linked by oxygen atoms. The crystalline framework contains open cavities in the form of cages and channels of discrete size and about 3 Å to about 30 Å in diameter. The primary building unit of a molecular sieve is the individual tetrahedral unit. The topology of all known molecular sieve framework types can be described in terms of a finite number of specific combinations of units.

Availability of the internal pore surface of zeolites is affected by the pore size of the zeolite. The size of the pore determines what the maximum allowable size is for molecules to enter. Pore size is determined by the ring size of the pore aperture and can also be affected by the size of the cation associated with the framework negative charge.

Examples of zeolites suitable for use in the present invention include small pore zeolites, medium pore zeolites, and large pore zeolites. A large pore zeolite generally has a pore size of >7 Å and includes, but is not limited to, zeolite types such as BEA, MAZ, MEI, FAU, and EMT. Examples of large pore zeolites include, but are not limited to, zeolite L (LTL), zeolite Y (FAU), zeolite X (FAU), offretite (OFF), omega, zeolite beta (BEA), mordenite (MOR), ZSM-3, ZSM-4, ZSM-18, and ZSM-20. A medium pore size catalyst generally has a pore size of at least about 5.5 Å; and generally the pore apertures consist of 10-membered ring structures and include types such as MFI, MEL, EUO, MTT, HEU, FER, and TON. Examples of medium pore zeolites include, but are not limited to, ZSM-5, ZSM-34, ZSM-38, and ZSM-48. A small pore size zeolite has a pore size from about 3 Å to about 5.0 Å. Generally, the pore apertures of the structure consist of from 8- to 10-membered ring structures and include, but are not limited to zeolite types such as CHA, ERI, KFI, LEV, and LTA. Examples of small pore zeolites include ZK-4, ZK-5, zeolite A, zeolite T, gmelinite, clinoptilolite, chabazite and erionite. Zeolite catalysts used as part of the invention also include gallosilicates and titanosilicates. Prior to using a zeolite molecular sieve catalyst of the invention, the zeolite catalyst can be calcined at a temperature of between about 500° C. to about 550° C.

Zeolite catalysts used as part of the invention preferably have a pore size with a diameter of at least about 3 Å. In one embodiment, the diameter of the pore of the zeolite catalyst is between about 3 Å to about 5 Å. In another embodiment, the diameter of the pore of the zeolite catalyst is between about 5 Å to about 7 Å. In yet another embodiment, the diameter of the pore of the zeolite catalyst is between about 7 Å to about 15 Å. In a further embodiment, the diameter of the pore of the zeolite catalyst is at least about 15 Å.

Further, zeolite catalysts useful with the invention can contain a Group I or II ion, although other metal, non-metal and organic cations can also balance the negative charge created by the presence of trivalent metals (e.g., aluminum (Al)) in the structure. In one aspect, the zeolite catalysts are in the acid form. As used herein, the “acid form” of the zeolite refers to a zeolite where the cations of the zeolite have been substituted with a proton. The acid form of the zeolites can be prepared by known methods. For example, H—Y zeolite can be prepared by ion exchange of Na—Y zeolite with 1M NH₄Cl of solution at 70° C. for 2 h with a subsequent calcination to temperatures in excess of 400° C.^{[13]} In one embodiment, the zeolite catalyst is H—Y zeolite. In another embodiment, the zeolite catalyst is H-ZSM. In yet another embodiment, the zeolite catalyst is H-zeolite beta (H-BEA or H-beta).

Non-zeolitic molecular sieves are microporous materials that are formed from aluminum oxide (AlO₂) and phosphorus oxide (PO₂) tetrahedra and have electrovalently neutral frameworks.^{[14]} A representative empirical formula of a non-zeolitic molecular sieves is:

(ELₓAl_{y}P_{z})O₂

where EL is an element selected from the group consisting of silicon, zinc, magnesium, iron, cobalt, nickel, manganese, chromium and mixtures thereof, x is the mole fraction of EL and is at least 0.005, y is the mole fraction of Al and is at least 0.01, z is the mole fraction of P and is at least 0.01, and x+y+z=1. When EL is a mixture of metals, x represents the total amount of the element mixture present. Non-zeolitic molecular sieves include, but are not limited to, aluminophosphates (AlPO₄)^{[15]}, silicoalumino-phosphates (SAPO), metalloaluminophosphates (MAPO), and nonmetal substituted aluminophosphates (NMAPO). Preferred elements (EL) are silicon, magnesium and cobalt, with silicon being especially preferred.

Non-zeolitic molecular sieves are generally synthesized by hydrothermal crystallization from a reaction mixture comprising reactive sources of aluminum, phosphorus, optionally one or more elements, other than aluminum and phosphorous, which are capable of forming oxides in tetrahedral coordination with AlO₂ and PO₂ units, and one or more organic templating agents.

The non-zeolite catalysts used as a part of the invention can have a pore size with a diameter of at least about 3 Å. For example, SAPO molecular sieves are generally classified as being microporous materials having 8, 10, or 12 membered ring structures. These ring structures can have an average pore size ranging from about 3.5 Å to about 15 Å. AlPO₄ molecular sieves typically have uniform pore dimensions ranging from about 3 Å to about 10 Å. In one embodiment, the diameter of the pore of the non-zeolite catalyst is between about 3 Å to about 5 Å. In another embodiment, the diameter of the pore of the non-zeolite catalyst is between about 5 Å to about 7 Å. In yet another embodiment, the diameter of the pore of the non-zeolite catalyst is between about 7 Å to about 15 Å. In a further embodiment, the diameter of the pore of the non-zeolite catalyst is at least about 15 Å.

Alumina is a highly porous material comprised of aluminum and oxygen with the chemical formula Al₂O₃. Examples of suitable alumina catalysts useful with the invention include, but are not limited to, alpha-alumina (α-Al₂O₃), beta-alumina (β-Al₂O₃), and gamma-alumina (γ-Al₂O₃) of various particle sizes. The alumina catalysts used in the methods of the current invention are acidic or contain acidic sites and have a particle size of at least about 0.5 microns.

Silica is a chemical compound of silicon and oxygen with the chemical formula SiO₂. The silica catalysts useful in the methods of the present invention are acidic or contain acid sites and have a particle size of at least 0.2 μm. Examples of suitable silica catalysts useful with the invention include, but are not limited to, MCM-4, SBA-15, and other related mesoporous silica catalysts.

Activated carbon catalysts for use with the present invention can have any shape such as granular, powdery, fibrous, sheet-like, or honeycomb-like shape and include, but are not limited to, powdered activated carbon (PAC), granular activated carbon (GAC), extruded activated carbon (EAC), bead activated carbon (BAC), impregnated carbon and polymer coated carbon. The activated carbons catalysts are generally derived from an organic source, such as wood, lignite, ground coconut shells, and coal. The forms of activated carbon include, but are not limited to, surface oxidized activated carbon, graphite, graphite oxide, and carbon nano-material (e.g., carbon nanotubes, carbon nanofibers). The activated carbon catalysts can be further modified to incorporate one or more surface modifications that allow control of the acidity or catalytic activity of the catalysts. The activated carbon catalysts can possess a specific surface area of at least 500 m²/g, preferably at least 750 m²/g, and more preferably at least 900 m²/g.

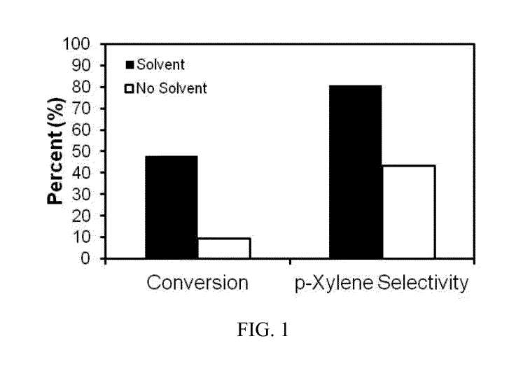

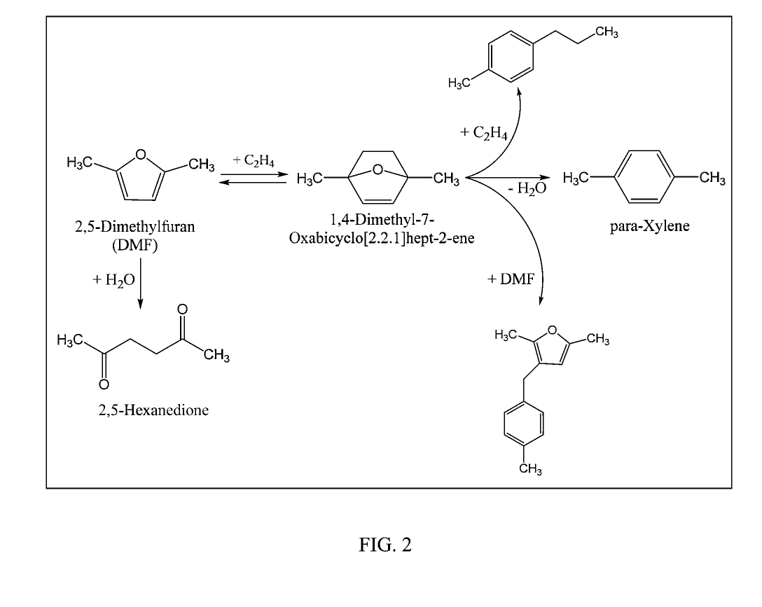

The methods of the present invention use a solvent for 2,5-dimethylfuran. As used herein, “solvent for 2,5-dimethylfuran” refers to solvents in which 2,5-dimethylfuran is partially or completely soluble. The presence of the solvent improves the conversion of 2,5-dimethylfuran to para-xylene and the selectivity for the formation of the desired p-xylene product minimizing the presence of undesired products that arise from competing side reactions. (See FIG. 1). Examples of competing side reactions that can arise when reacting 2,5-dimethylfuran and ethylene under cycloaddition conditions include hydrolysis of 2,5-dimethylfuran to 2,5-hexanedione, secondary addition of 2,5-dimethylfuran to produce dimers, and secondary reactions of ethylene to produce alkylated aromatics. (See FIG. 2). As used herein, “improvement in selectivity” to para-xylene refers to the increased quantity of carbon derived from 2,5-dimethylfuran being converted to the product, para-xylene.

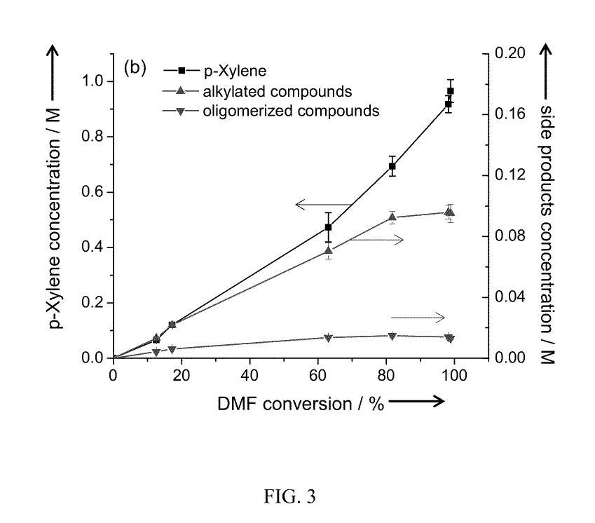

As shown in FIG. 3, selectivity for the formation of para-xylene from the reaction of 2,5-dimethylfuran in heptane with ethylene at 250° C. in the presence of acidic zeolite beta (H-BEA or H-beta) catalyst increases with 2,5-dimethylfuran conversion. At 20% conversion, selectivity to para-xylene is only 60%, and the hydrolysis product (2,5-hexanedione), alkylated products and oligomers comprise the remaining 40%, within experimental error. However, as conversion increases to >99%, selectivity to para-xylene exceeds 90%.

The solvent used in the methods of the present invention can be polar or non-polar. As used herein, “non-polar solvents” refers to solvents with a dielectric constant of less than 15 and can include, but are not limited to, aliphatic solvents, aromatic solvents, and other aprotic solvents. Examples of non-polar solvents include, but are not limited to, pentane, hexanes, heptane, hexadecane, cyclohexane, benzene, toluene, xylene, tetrahydrofuran, diethylether, ethyl acetate, and methylene chloride. In one embodiment, the non-polar solvent is an aliphatic solvent. In another embodiment, the non-polar solvent is an aromatic solvent. In yet another embodiment, the non-polar solvent is an aprotic solvent.

“Aliphatic solvents” or “(C5-C20) aliphatic solvents,” as used herein, refer to straight chained, branched or non-aromatic cyclic (C5-C20) hydrocarbon containing solvents. Examples of aliphatic solvents include but are not limited to, pentane, hexane, heptane, hexadecane, and cyclohexane. “Aromatic solvents” or “(C6-C18) aromatic solvents” refer to cyclic aromatic (C6-C 18) hydrocarbon containing solvents. Examples of aromatic solvents include, but are not limited to, benzene, toluene, and xylene. “Aprotic solvents,” as used herein, refer to solvents that do not yield or accept a proton. Examples of aprotic solvents include, but are not limited to, tetrahydrofuran, diethylether, ethyl acetate, and methylene chloride.

“Polar solvents,” as used herein, refer to solvents with a dielectric constant of more than 15 and include, but are not limited to, aprotic and protic solvents. Examples of polar solvents include, but are not limited to, acetone, dimethylformamide, acetonitrile, propylene carbonate, acetic acid, formic acid, methanol, ethanol, n-propanol, isopropanol, n-butanol, nitromethane, and water. In one embodiment, the polar solvent is an aprotic solvent. In another embodiment, the polar solvent is a protic solvent. “Protic Solvents,” as used herein, refer to solvents capable of yielding or accepting a proton.

The amount of solvent present in the reaction mixture comprises at least 20 wt % of the reaction mixture. As used herein, “percentage of the reaction mixture” refers to the weight of solvent present as compared to the total weight of 2,5-dimethylfuran and solvent present in the reaction mixture expressed as a percentage. In one embodiment, the amount of solvent comprises at least 20 wt % of the reaction mixture. In another embodiment, the amount of solvent comprises at least about 50 wt % of the reaction mixture. In yet another embodiment, the amount of solvent comprises from at least about 75 wt % of the reaction mixture.

The methods of the present invention are conducted at an elevated temperature and an elevated pressure. In one embodiment, the elevated pressure of ethylene is at least about 5 bar (73 psi). In another embodiment, the elevated pressure of ethylene is at least about 20 bar (290 psi). In yet another embodiment, the elevated pressure of ethylene is at least about 40 bar (580 psi).

The reaction is also conducted at an elevated temperature, preferably at least about 100° C., more preferably at least about 200° C., and most preferably at least about 250° C.

The methods of the present invention include a reaction time of at least one minute. In one embodiment, the reaction time is at least about 1 minute. In another embodiment, the reaction time is at least about 1 hour. In yet another embodiment, the reaction time is at least about 6 hours. In further embodiment, the reaction time is at least about 12 hours. In another embodiment, the reaction time is at least about 24 hours. In yet another embodiment, the reaction time is at least about 48 hours.

The methods of the present invention can be carried out in a variety of reactor types. Examples of reactors than can be used in the present invention include, but are not limited to, trickle bed reactors, slurry reactors, multiphase reactors, microreactors, fixed-bed reactors and fluid-bed reactors.

The methods of the present invention can provide conversions of 2,5-dimethylfuran of at least about 5% and yields of at least about 60%. Depending on the identity of the catalyst, the temperature and the solvent conversions of 2,5-dimethylfuran can be preferably at least about 5%, more preferably at least 50%, even more preferably at least about 90%. As defined herein, “yield” refers to the amount of para-xylene (in moles) obtained from the cycloaddition reaction based on the amount (in moles) of 2,5-dimethylfuran (e.g., limiting reagent/reactant) used. Generally, the yields of para-xylene are at least about 60%, preferably at least about 80% and more preferably at least about 90%.

Further, the methods of the present invention can provide para-xylene with a selectivity of at least about 60%. In one embodiment, the selectivity for the formation of para-xylene is at least about 70%. In another embodiment, the selectivity for the formation of para-xylene is at least about 80%. In yet another embodiment, the selectivity for the formation of para-xylene is at least about 90%. In a further embodiment, the selectivity for the formation of para-xylene is at least about 95%.

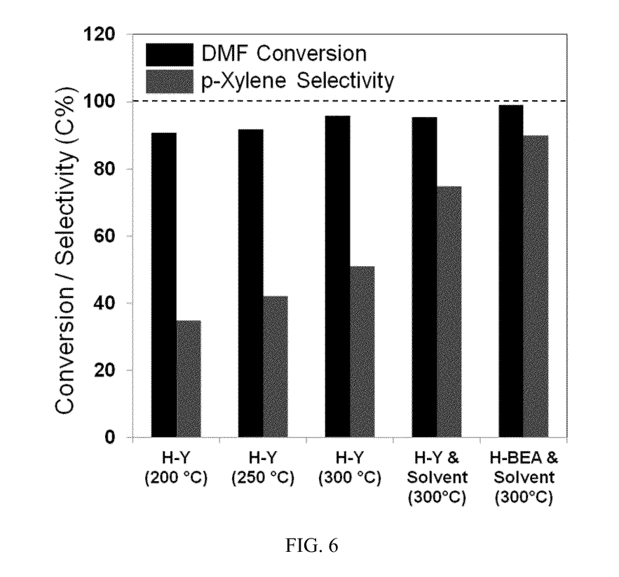

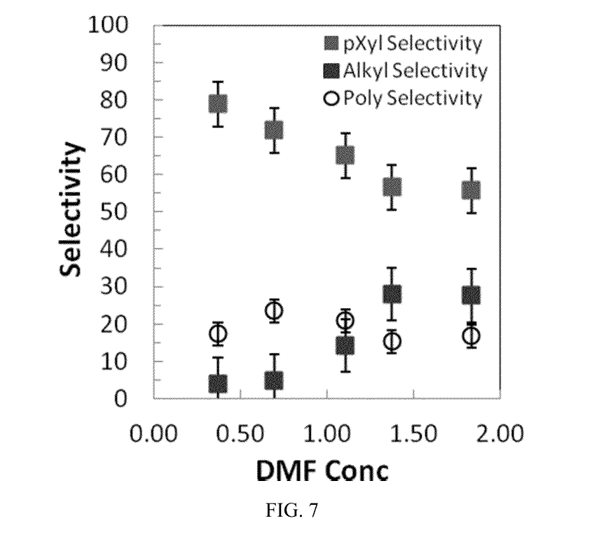

As shown in FIG. 6, increasing the temperature can increase para-xylene selectivity from about 35% at 200° C. to about 50% when pure 2,5-dimethylfuran (DMF) is treated with ethylene at 14 bar (200 psig) and H—Y zeolite catalyst at 300° C. Addition of the aliphatic solvent, n-heptane, can further increase selectivity to about 75%. Substitution of an H—Y zeolite catalyst with an H-beta zeolite catalyst can improve the selectivity for the formation of para-xylene to 90% upon reaction of 2,5-dimethylfuran in heptane with ethylene at 300° C. (FIG. 6). Changes in selectivity were also observed at low catalyst loading of H-beta zeolite catalyst and increasing concentrations of 2,5-dimethylfuran. As the concentration of 2,5-dimethylfuran was increased from 0.3M to 1.7M, para-xylene selectivity decreased from about 80% to about 57% while alkylated products increased and polymerized products remained constant. (FIG. 7)

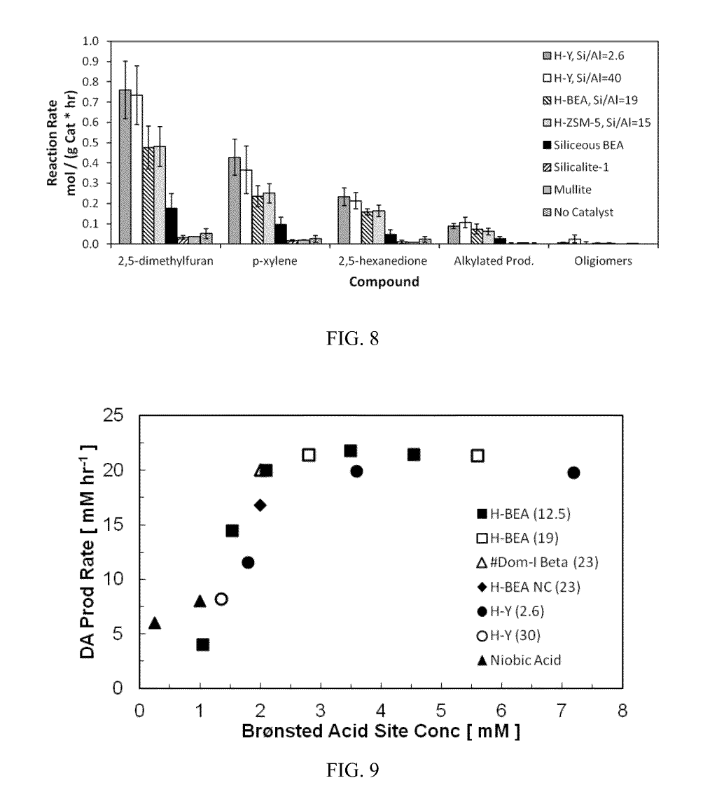

The initial reaction rate for the conversion of 2,5-dimethylfuran (X_{2,5}-diemethylfuran<10%) and the product formation can vary depending on the catalyst microstructure and active site. Reaction rates of siliceous catalysts and catalysts containing Brønsted acid sites were measured using pure 2,5-dimethylfuran with the continuous addition of ethylene (57 bar) at 300° C. (FIG. 8) Catalysts that contain a Brønsted acid site showed much greater activity than siliceous materials that did not indicating that Brønsted acid sites are necessary to promote the dehydration of the cycloadduct.

FIG. 9 shows the existence of two distinct reaction regimes for a variety of Brønsted acid catalysts as the catalyst loading increases. Increasing catalyst loading changes the Brønsted acid site loading. As the catalyst loading increases and the acid site concentration exceeds 2.5 mM, a second distinct reaction regime appears as illustrated in FIG. 9.

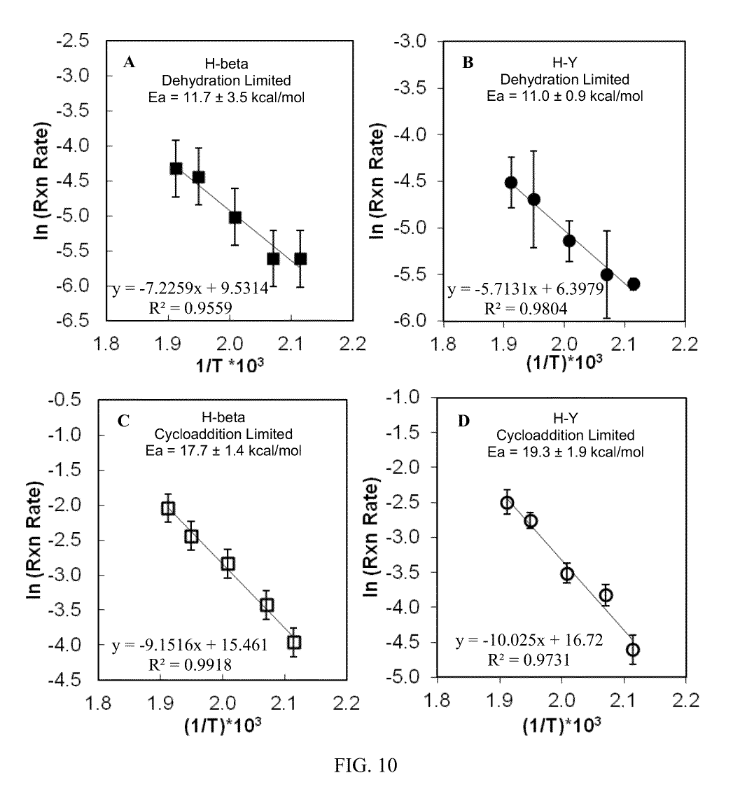

FIG. 10 shows the experimentally measured activation energies for both an H-beta zeolite catalyst and an H—Y zeolite catalyst at low and high catalyst loading. Measurement of the activation energy in each regime provided the rate-controlling step for that regime. The low catalyst loading or “dehydration limited” activation energies were measured at a catalyst loading of about 1.25 mM and the high catalyst loading or “cycloaddition limited” activation energies were measured at about 3.25 mM for both the H-beta and the H—Y zeolite catalysts. The dehydration limited (low catalyst loading) activation energy was determined to be 11.7±3.5 kcal/mol for the H-beta zeolite and 11.0±0.9 kcal/mol for the H—Y zeolite catalyst is 11.0±0.9 kcal/mol. The cycloaddition limited (high catalyst loading) activation energy was determined to be 17.7±1.4 kcal/mol for the H-beta zeolite catalyst and 19.3±1.9 kcal/mol for the H—Y zeolite catalyst. (FIG. 10). (FIG. 10) The measured activation energies confirmed the existence of the two proposed reaction regimes.

The reaction orders for both 2,5-dimethylfuran and ethylene measured in the cycloaddition limited regime indicated that changing 2,5-dimethylfuran concentration affects the overall reaction rate. The cycloaddition limited reaction order for 2,5-dimethylfuran is 0.87±0.03 (FIG. 11A) and for ethylene is 1.10±0.07 (FIG. 11B). A reaction order greater than zero for 2,5-dimethylfuran indicates that the reaction does not proceed directly from saturated catalyst active sites.

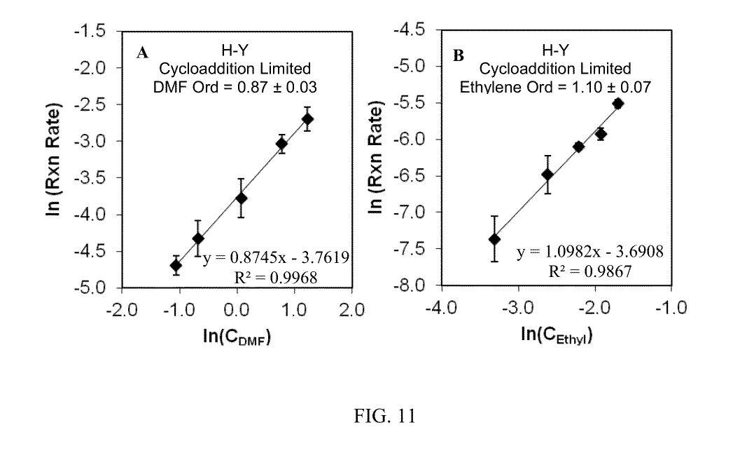

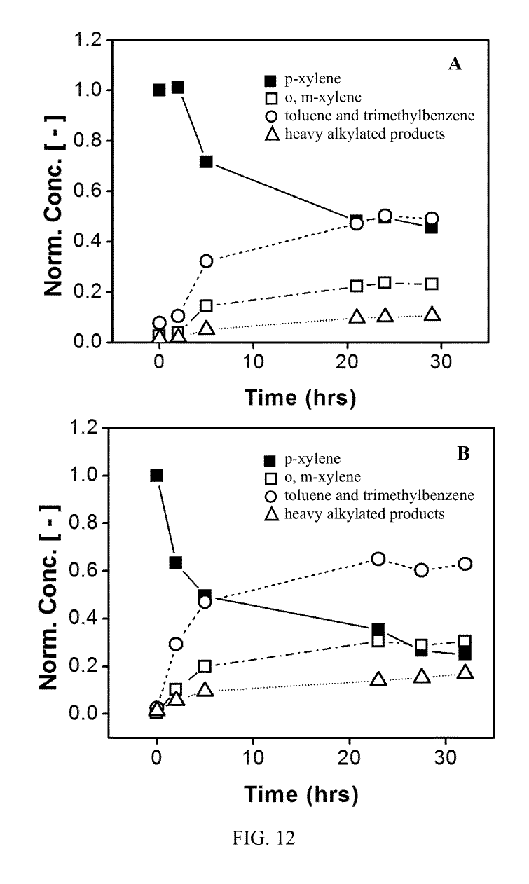

Isomerization of para-xylene was observed to readily occur at conditions relevant to production of para-xylene from 2,5-dimethylfuran and ethylene. The extent of isomerization was demonstrated to be dependent upon the catalyst loading suggesting catalyst deactivation. This resulted in non-equilibrated products of p-xylene including ortho, meta-xylene (o, m-xylene), toluene and trimethylbenzene, and alkylated products. (FIG. 12)

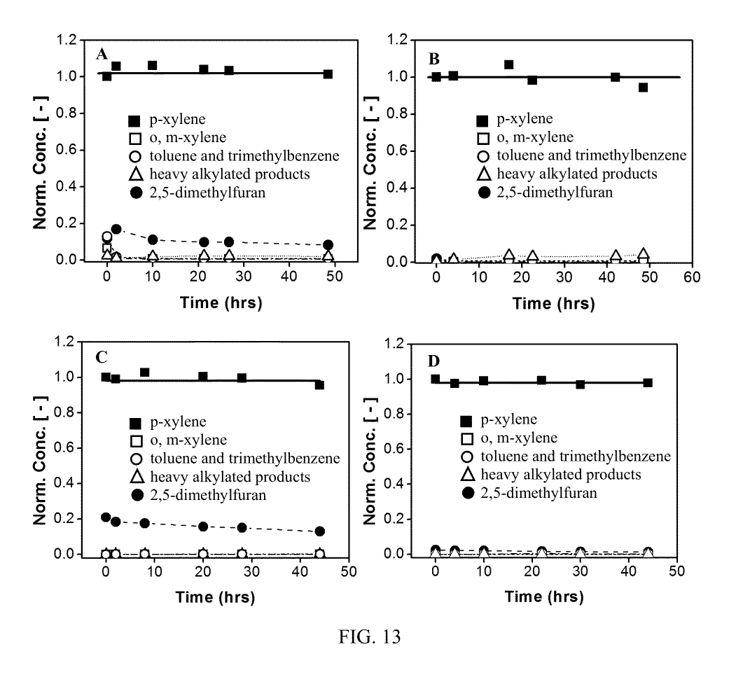

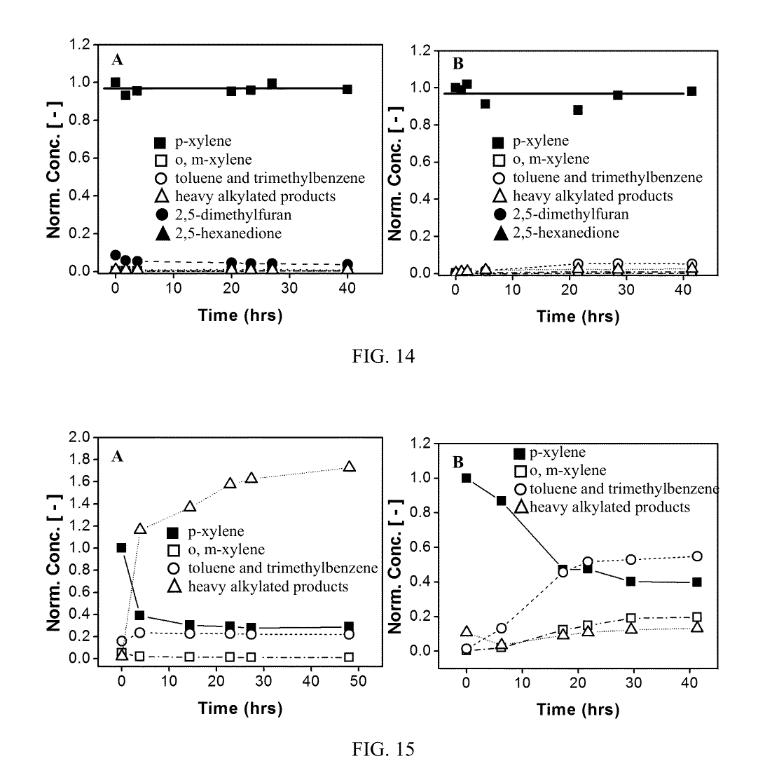

2,5-Dimethylfuran was observed to inhibit the isomerization of para-xylene in the presence of an H—Y or an H-beta zeolite catalyst. 2,5-dimethylfuran inhibits para-xylene isomerization when present at ⅕ the concentration of para-xylene. Inhibition of para-xylene isomerization was also observed at concentrations of 2,5-dimethylfuran as low as 1/50 the concentration of para-xylene. (FIG. 13) As illustrated in FIG. 14, the presence of 2,5-hexanedione can also inhibit isomerization of para-xylene in the presence of an H—Y zeolite catalyst. 2,5-hexanedione was shown to inhibit para-xylene isomerization when present at both ⅕ and 1/50 the concentration of para-xylene. (FIG. 14)

Experiments were also carried out to determine the effect of the presence of ethylene and water on para-xylene isomerization. FIG. 15A shows that ethylene in the presence of H—Y zeolite catalyst, heptane and para-xylene can promote the formation of heavy alkylated products in addition to isomerization of para-xylene to o,m-xylene, toluene and trimethylbenzene. FIG. 15B shows that the presence of water does not inhibit para-xylene isomerization.

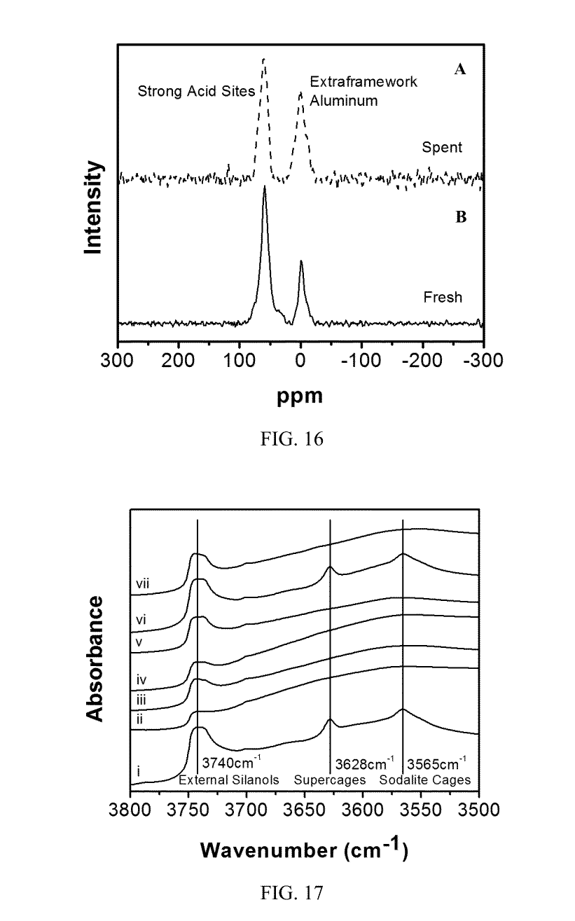

FIG. 16 shows an ²⁷Al-NMR spectra of fresh, unused H—Y zeolite catalyst and of spent H—Y zeolite catalyst that has been used in a cycloaddition reaction and not regenerated by coke burnoff. The ²⁷Al-NMR of the spent H—Y zeolite catalyst shows a reduction of tetrahedrally-coordinated aluminum (ca. 60 ppm) relative to octahedrally-coordinated aluminum (ca. 0 ppm) when compared to unused, fresh H—Y zeolite catalyst. (FIG. 16B)

FIG. 17i shows the diffuse reflectance of a degassed H—Y zeolite catalyst. Degassed zeolite H—Y (Si/Al2.6) exhibits —OH stretches associated with external silanols (3740 cm⁻¹) and Brønsted acid sites in supercages (3628 cm⁻¹) and sodalite cages (3565 cm⁻¹). The DRIFT spectra also showed that as 2,5-dimethylfuran (FIG. 17ii), para-xylene (FIG. 17ii), or mixtures of 2,5-dimethylfuran/para-xylene (FIG. 17iv) were adsorbed onto the zeolite catalyst at 120° C., the peaks associated with the Brønsted acid sites disappear. Increasing the temperature to 300° C. recovered Brønsted acids previously occupied by para-xylene (FIG. 17iv), while 2,5-dimethylfuran remained adsorbed on Brønsted acid sites associated within super- and sodalite cages. (FIGS. 17v and 17vii).

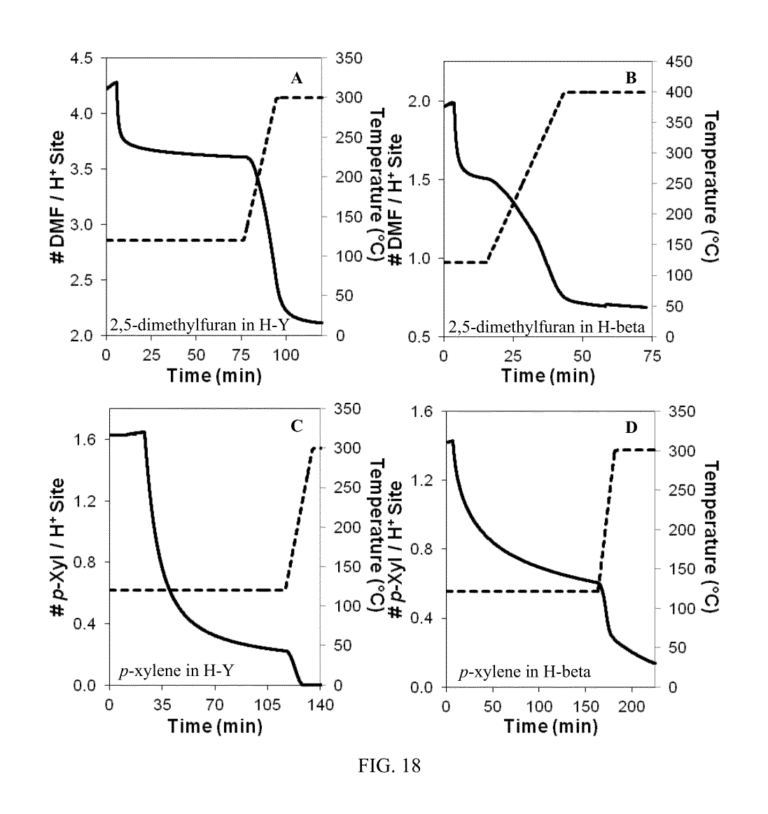

Thermogravimetric analysis (TGA) of adsorbed 2,5-dimethylfuran on H—Y (FIG. 18A) and H-beta zeolite (FIG. 18B) shows incomplete desorption. In comparison, TGA of para-xylene on H—Y (FIG. 18C) and H-beta (FIG. 18D) shows complete desorption at 300° C.

### EXEMPLIFICATION

### Example 1

### Comparison of Conversion and Selectivity in the Production of Para-Xylene in the Presence of H-Beta Catalyst with and without n-Heptane Solvent

Experiments for the production of para-xylene with solvent were conducted in a 160 ml batch reactor with a temperature controller and equipped with a gas entrainment impellor, allowing for fast mass transfer of ethylene (k_{L}a=6.16 hr⁻¹). The reaction solution consisted of 15 ml of 2,5-dimethylfuran (18.6 wt %) in 83 ml n-heptane (81.4 wt %) with 0.45±0.05 g of H-beta zeolite catalyst and 2 ml n-tridecane used as an internal standard. The H-beta zeolite catalyst was calcined at 550° C. for 12 hours in a tube furnace prior to use in the cycloaddition reaction.

Experiments for the production of para-xylene without solvent were carried out in a 100 ml reaction vessel with a temperature controller and equipped with a gas entrainment stirrer. The reaction solution consisted of 49 ml of 2,5-dimethylfuran (98.3 wt %) with 0.30±0.05 g of H-beta zeolite catalyst and 1 ml n-tridecane used as an internal standard. The H-beta zeolite catalyst was calcined at 550° C. for 12 hours in a tube furnace prior to use in the cycloaddition reaction.

For experiments either in the presence or in the absence of solvent, the reaction solution was placed in the reaction vessel. The reaction vessel was purged with nitrogen and then stirred at a rate of 1000±100 rpm while heating to 300° C. Once the desired temperature was achieved, approximately 14 bar partial pressure of ethylene gas was added to the reactor. Samples were collected immediately after ethylene addition and at later times. Subsequent samples were taken periodically under the reaction conditions over a period of 24 hours using a double block sample system. An emphasis was placed on early kinetics and the data utilized to compare solvated (with solvent) and unsolvated (without solvent) experiments was collected at a reaction time of 2 hours.

Liquid samples were analyzed using an Agilent 6890 gas chromatography system equipped with a flame ionization detector, a G133A autosampler, and 30 meter HP-Innowax column (to ensure the separation of para and meta xylenes). All reactions were run at least three times, and error bars were calculated in a 90% confidence interval. (See FIG. 1). As illustrated in FIG. 1, a substantial increase in both conversion and selectivity for the formation of para-xylene is observed upon inclusion of a solvent as compared to experiments run in the absence of a solvent.

### Example 2

### Production of Para-Xylene Using Acidic Zeolite Beta (H-Beta) Catalyst

Experiments were conducted in a 160 ml batch reactor equipped with a gas entrainment impellor, allowing for fast mass transfer of ethylene (k_{L}a=6.16 hr⁻¹). The reaction solution consisted of 100 ml of 2,5-dimethylfuran in heptane (18.5 wt % of 2,5-dimethylfuran/81.5 wt % heptane) with 0.45±0.05 g of H-beta catalyst and n-tridecane used as an internal standard. The zeolite H-beta catalyst was calcined at 550° C. for 12 hours in a tube furnace prior to use in the cycloaddition reaction.

The reactor solution was placed in the reaction vessel. The reaction vessel was purged with nitrogen and then stirred at a rate of 950±50 rpm while heating to 250° C. Once the reaction temperature was achieved, approximately 14 bar partial pressure of ethylene gas was added to the reactor. Samples were collected immediately after ethylene addition and at later times. Subsequent samples were taken periodically over a period of 24 to 48 hours. All reactions were run at least three times, and error bars were calculated in a 90% confidence interval. Liquid samples were analyzed using a gas chromatography system equipped with a flame ionization detector. Conversion to para-xylene of >99% was observed with >90% selectivity. (See FIG. 3 & FIG. 4).

### Example 3

### Production of Para-Xylene Using Acidic Zeolite H—Y Catalyst

The reaction was carried out in a 100 mL, high-pressure, high temperature benchtop reactor with a temperature controller. 2,5-Dimethylfuran (reactant, 30.2 wt % in 69.8 wt % heptane) was reacted with H—Y zeolite catalyst over a temperature range of 200 to 300° C. with a loading of 0.25 g of catalyst to 49 mL of reactant and 1 mL of n-tridecane as an internal standard. The reaction vessel was purged with N₂ and stirred at 1000 rpm with a gas entrainment impeller while heating to the desired temperature.

Once the reaction temperature was achieved, the vessel was pressurized to 57 bar (825 psi) with ethylene gas, which was continuously added during the reaction. Samples were collected immediately after ethylene addition and at later times corresponding to a 2,5-dimethylfuran conversion of about 10% while the system was at reaction conditions. Products were identified using a gas chromatograph/mass spectrometer and by comparing retention times with those of pure standards. Quantification was performed using a gas chromatograph equipped with a flame ionization detector.

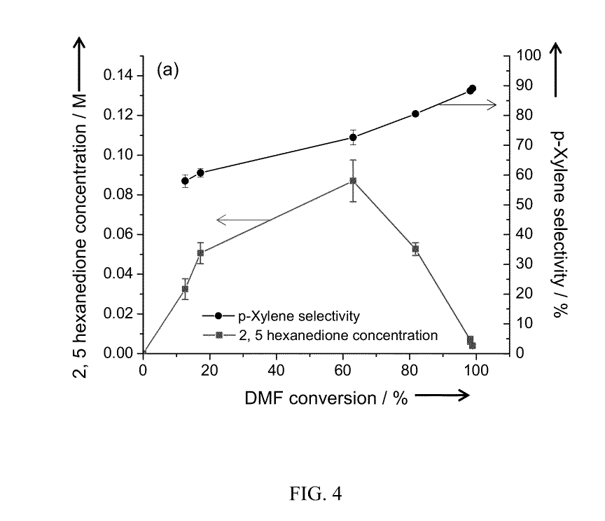

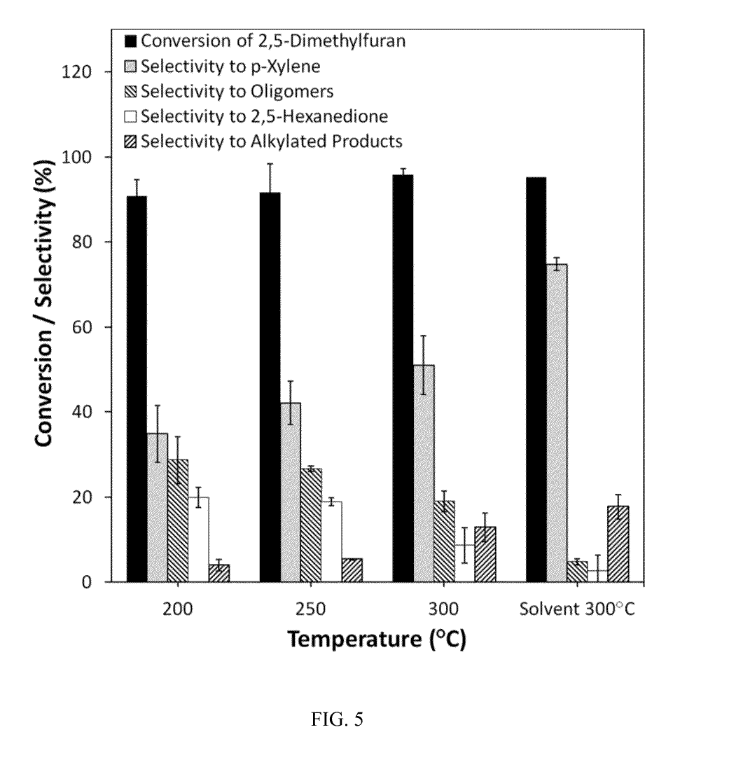

At 30.2 wt % dimethylfuran in n-heptane, ninety-five percent (95%) conversion of 2,5-dimethylfuran was observed with H—Y zeolite catalyst (Si/Al=30) at 300° C. with 75.7±1.5% selectivity for the formation of para-xylene. (See FIG. 5).

### Example 4

### Production of Para-Xylene Using γ-Al₂O₃ Catalyst

Experiments were conducted in a 160 ml batch reactor equipped with a gas entrainment impellor, allowing for fast mass transfer of ethylene (k_{L}a=−6.16 hr⁻¹). Gamma alumina (γ-Al₂O₃) was calcined at 500° C. for 12 hours prior to use. The reaction solution consisted of 100 ml of 2,5-dimethylfuran in heptane (18.5 wt % of 2,5-dimethylfuran/81.5 wt % heptane) with 0.45±0.05 g of γ-Al₂O₃ catalyst and n-tridecane used as an internal standard.

The reactor solution was placed in the reaction vessel. The reaction vessel was purged with nitrogen and then stirred at a rate of 950±50 rpm while heating to 250° C. Once the reaction temperature was achieved, approximately 14 bar partial pressure of ethylene gas was added to the reactor. Samples were collected immediately after ethylene addition and at later times. Subsequent samples were taken periodically over a period of 24 to 48 hours. All reactions were run at least three times, and error bars were calculated in a 90% confidence interval. Liquid samples were analyzed using a gas chromatography system equipped with a flame ionization detector. Conversion of 60% to para-xylene was observed over a reaction time of 30 hours.

### Example 5

### Characterization of Brønsted Acid Sites of Zeolite Molecular Sieve Catalysts

The number of Brønsted acid sites was characterized by temperature programmed desorption coupled with thermal gravimetric analysis (TPD-TGA) on a thermal analyzer (SDT Q600, TA) for isopropylamine (IPA). The TGA plots indicate a two-step weight change for the samples. The weight loss at low temperature (<300° C.) is attributed to desorption of physisorbed IPA, whereas the one between 300° C. and 400° C. is from the decomposition of IPA on strong Brønsted acid sites.^{[16]}

### Example 6

### Determination of Brønsted Acid Sites of Zeolite Molecular Sieve Catalysts

The samples were outgassed at 400° C. under constant helium flow. The ammonia flowed through the sample bed and adsorbed at 100° C. for 30 minutes. After saturation, the sample was purged with helium again to remove excess and weakly adsorbed ammonia from the sample. Then the sample was heated to 700° C. with a ramping rate of 10° C. per minute and data were collected during the heating period. The number of total acid sites was determined by using ammonia temperature programmed desorption (NH₃-TPD) with an automatic chemisorption analyzer (ChemBET Pulsar™ TPR/TPD, Quantachrome) coupled with a thermal conductivity detector (TCD).

### Example 7

### Comparison of Conversion and Selectivity in the Production of Para-Xylene in the Presence of H-Beta and H—Y Catalyst with and without n-Heptane Solvent

Experiments for the production of para-xylene using a zeolite catalyst and without solvent were conducted in a 160 ml reaction vessel. The reaction solution consisted of 98 ml of pure 2,5-dimethylfuran, 0.5 g of a zeolite catalyst and 2 ml tridecane used as internal standard. The reaction vessel was sealed and purged with N₂ and stirred with a gas entrainment stirrer at 1000 rpm to ensure adequate mass transfer of ethylene gas to the reaction solution. Upon reaching reaction temperature between 200 and 300° C., an additional 14 bar (200 psig) of ethylene was added to start the reaction. Experiments comparing para-xylene yield were run for 24 to 48 hrs and sampled in approximately 8 hour increments using a double block sampling system to monitor reaction progress. Comparison across changing temperature and catalyst was done at conversion exceeding 90%.

Experiments for the production of para-xylene using a zeolite catalyst and with a solvent were conducted in a manner identical to that of pure 2,5-dimethylfuran without solvent except for the dilution of the 2,5-dimethylfuran with the desired solvent. The reaction solution for the solvated experiments consisted of 15 ml of 2,5-dimethylfuran (18.6 wt %) in 83 ml n-heptane (79.3 wt %), 0.5 g of a zeolite catalyst (H—Y or H-beta zeolite catalyst), and 2 ml tridecane (2.1 wt %) as an internal standard at 300° C. Comparison across catalysts was done at conversion exceeding 90%.

For experiments either in the presence or in the absence of solvent, the reaction solution was placed in the reaction vessel. The reaction vessel was purged with nitrogen and then stirred at a rate of 1000±100 rpm while heating. Once the desired temperature was achieved, approximately 14 bar (200 psi) partial pressure of ethylene gas was added to the reactor. Samples were collected immediately after ethylene addition and at later times. Subsequent samples were taken periodically under the reaction conditions over a period of 24 hours using a double block sample system. An emphasis was placed on long term conversion and selectivities and the data utilized to compare solvated (with solvent) and unsolvated (without solvent) experiments was collected over the course of 24 h to 48 h.

Liquid samples were analyzed using an Agilent 6890 gas chromatography system equipped with a flame ionization detector, a G133A autosampler, and 30 meter HP-Innowax column (to ensure the separation of para and meta xylenes). All reactions were run at least three times, and error bars were calculated in a 90% confidence interval. (See FIG. 6). As illustrated in FIG. 6, a substantial increase in both conversion and selectivity for the formation of para-xylene was observed upon inclusion of a solvent as compared to experiments run in the absence of a solvent.

### Example 8

### Comparison of Selectivity in the Production of Para-Xylene in the Presence of H-Beta with Increasing 2,5-Dimethylfuran Concentration

Experiments were carried out in a 160 ml reactor. The reaction solution containing a total volume of 100 ml consisted of 2,5-dimethylfuran in heptane, and 2.1% n-tridecane (as an internal standard). 0.22 g of H-beta zeolite catalyst (Si/Al=12.5) was added to the reaction solution and the reactor was purged with N₂ and heated to 200° C. Once at 200° C., 14 bar (200 psig) of ethylene gas was added. The reaction solution composition changed based on the amount of 2,5-dimethylfuran. The concentration of 2,5-dimethylfuran was varied between about 6.4 wt % to about 30.2 wt % in n-heptane solvent. Selectivities were measured using the initial kinetics data for the formation of all Diels-Alder products. Samples were collected immediately after ethylene addition and at later times. Subsequent samples were taken periodically over the first hour of reaction.

Liquid samples were analyzed using an Agilent 6890 gas chromatography system equipped with a flame ionization detector, a G133A autosampler, and 30 meter HP-Innowax column (to ensure the separation of para and meta xylenes). A decrease in para-xylene selectivity was observed as the concentration of 2,5-dimethylfuran was increased as can be seen in FIG. 7.

### Example 9

### Comparison of Initial Reaction Rates of 2,5-Dimethylfuran Cycloaddition with Ethylene Using Various Catalysts

Experiments comparing the initial reaction rate of various catalysts were conducted using pure 2,5-dimethylfuran. To a reactor was added 98 ml of 2,5-dimethylfuran, 2 ml of tridecane (as an internal standard), and 0.45 g of the desired catalyst. The reaction vessel was then purged with N₂ and heated to a temperature of 300° C. while stirring using a gas entrainment impeller. The use of a gas entrainment helps eliminate mass transfer limitations. Once at 300° C., 14 bar (200 psig) of ethylene was added for a total vessel pressure of 57 bar (800 psi). Samples were taken periodically under the reaction conditions from the start of the reaction until about 10% conversion using a double block sampling system. Conversion was maintained below 10% for accurate initial kinetics measurements.

Liquid samples were analyzed using an Agilent 6890 gas chromatography system equipped with a flame ionization detector, a G133A autosampler, and 30 meter HP-Innowax column (to ensure the separation of para and meta xylenes). As can be seen in FIG. 8, catalysts that contained Brønsted acid sites showed much greater activity than siliceous materials lacking Brønsted acid sites.

### Example 10

### Study of the Effect of the Brønsted Acid Site Concentration on the Reaction Regime of the 2,5-Dimethylfuran Cycloaddition with Ethylene

Experiments for studying the effect of Brønsted acid site concentration were conducted by placing 15 ml of 2,5-dimethylfuran (18.6 wt %) in 83 ml n-heptane (79.3 wt %), and 2 ml of tridecane (2.1 wt %) used as an internal standard. The Brønsted acid catalyst was then added and the resulting mixture was purged with N₂ and heated to 200° C. Once at 200° C., the reaction vessel pressurized to 38 bar (550 psig) with ethylene gas. Catalyst loading was varied between 0.25 mM to 7.5 mM acid site concentration. Since acid site density changes from catalyst to catalyst, the actual mass of catalyst varied based on the catalyst type and desired effective acid site concentration. The results are shown in FIG. 9. As the Brønsted acid site concentration was increased, a second distinct reaction regime was observed. (FIG. 9)

### Example 11

### Measurement of the Activation Energies of the Cycloaddition Limited and Dehydration Limited Reaction Between 2,5-Dimethylfuran and Ethylene Using H—Y and H-Beta Zeolite Catalysts

Experiments for the measurement of the activation energy of the cycloaddition limited and dehydration limited regimes were conducted by placing 15 ml of 2,5-dimethylfuran (18.6 wt %) in 83 ml of n-heptane (79.3 wt %), and 2 ml of tridecane (2.1 wt %) (as an internal standard) in a reaction vessel. The zeolite catalyst was then added (H—Y or H-beta zeolite catalyst) and the resulting mixture was purged with N₂ and heated to 200° C. Once at 200° C., the reaction vessel pressurized to 14 bar (200 psig) with ethylene gas. Catalyst loading was 1.25 mM and 3.25 mM for the dehydration and cycloaddition limited regimes, respectively. Reaction rates were measured for the formation of all Diels-Alder products over the first hour of reaction time starting as soon as reaction temperature was reached and ethylene gas was added.

Liquid samples were analyzed using an Agilent 6890 gas chromatography system equipped with a flame ionization detector, a G133A autosampler, and 30 meter HP-Innowax column (to ensure the separation of para and meta xylenes). The measured activation energies for both the cycloaddition limited and the dehydration limited regimes in the presence of an H—Y and an H-beta zeolite catalyst are shown in FIGS. 10A and 10B.

### Example 12

### Measurement of the Reaction Order of 2,5-Dimethylfuran and Ethylene in the Cycloaddition Limited Regime Using H—Y Zeolite Catalyst

The measured reaction orders for 2,5-dimethylfuran and ethylene were done using H—Y zeolite at an effective acid site concentration of 3.25 mM so as to be in the cycloaddition reaction limited regime.

Experiments for the study of the reaction order of 2,5-dimethylfuran in the cycloaddition limited regime were conducted at a constant pressure of ethylene of 14 bar (200 psig) and at temperature of 200° C. A reaction solution containing a total volume of 100 ml consisting of 2,5-dimethylfuran in n-heptane solvent and 2.1 wt % tridecane used as an internal standard was placed in a reactor. H—Y zeolite catalyst (3.25 mM) was then added and the resulting mixture reaction vessel was purged with N₂ and heated to 200° C. Once at 200° C., 14 bar (200 psig) of ethylene was added. 2,5-Dimethylfuran concentration was varied from 6.4% to 30.2 wt % (0.3M to 3.3 M) in n-heptane solvent. The reaction rate for the formation of all Diels-Alder products was measured over the course of the first hour starting as soon as reaction temperature was reached and ethylene gas was added.

Experiments for the study of the reaction order of ethylene in the cycloaddition limited regime were conducted by placing 18.5 wt % (1.4 M) 2,5-dimethylfuran in 83 ml n-heptane (79.3 wt %), and 2 ml tridecane (2.1 wt %) used as an internal standard in a reaction vessel. H—Y zeolite catalyst (3.25 mM) was then added and the resulting mixture reaction vessel was purged with N₂ and heated to 200° C. Once at 200° C., ethylene was added and the concentration of ethylene was the increased from 0.04M to 0.18M by increasing ethylene pressure from about 7 bar to about 34 bar (100 psig to 500 psig). The reaction rate for the formation of all Diels-Alder products was measured over the course of the first hour starting as soon as reaction temperature was reached and ethylene gas was added.

Liquid samples were analyzed using an Agilent 6890 gas chromatography system equipped with a flame ionization detector, a G133A autosampler, and 30 meter HP-Innowax column (to ensure the separation of para and meta xylenes). The results are shown in FIGS. 11A and 11B. The reaction order obtained for 2,5-dimethylfuran was greater than zero indicating that the cycloaddition reaction does not proceed directly from saturated catalyst sites.

### Example 13

### Study of the Effect of the Reaction Conditions on Para-Xylene Isomerization in the Absence of 2,5-Dimethylfuran and Ethylene

To the reaction vessel was added 12 ml of para-xylene, 86 ml of n-heptane, and 2 ml of tridecane (as an internal standard). 0.45 g or 1.45 g H—Y zeolite catalyst was then added to the reaction vessel depending on the experiment. After the catalyst was added, the reaction vessel was purged with N₂ and heated to a reaction temperature of 300° C. Once at 300° C., 14 bar (200 psi) of N₂ was added to aid sampling and replenished as samples were taken. Samples were taken periodically under the reaction conditions over the course of 24 to 48 hours using a double block sampling system.

Liquid samples were analyzed using an Agilent 6890 gas chromatography system equipped with a flame ionization detector, a G133A autosampler, and 30 meter HP-Innowax column (to ensure the separation of para and meta xylenes). As shown in FIGS. 12A and 12B, isomerization of para-xylene was observed to readily occur at conditions relevant to the production of para-xylene from 2,5-dimethylfuran and ethylene.

### Example 14

### Study of the Effect of the Change in Concentration of 2,5-Dimethylfuran on the Inhibition of Para-Xylene Isomerization

para-Xylene isomerization was inhibited by the addition of 2,5-dimethylfuran in heptane solvent. The inhibition effects of 2,5-dimethylfuran were tested at various concentrations of 2,5-dimethylfuran.

2 ml of 2,5-dimethylfuran, 10 ml of para-xylene, 86 ml of n-heptane, and 2 ml of tridecane (as an internal standard) were placed in a reaction vessel. 0.45 g of H—Y zeolite was then added and the flask was purged with N₂ and heated to about 300° C. Once at 300° C., 14 bar (200 psi) of N₂ was added to aid sampling and replenished as samples were taken. Samples were taken periodically under the reaction conditions over the course of 24 to 48 hours using a double block sampling system.

Alternatively, 0.2 ml of 2,5-dimethylfuran, 12 ml of para-xylene, 86 ml of n-heptane, and 2 ml of tridecane used as an internal standard were placed in a reaction vessel. 0.45 g of H—Y zeolite was then added and the flask was purged with N₂ and heated to about 300° C. Once at 300° C., 14 bar (200 psi) of N₂ was added to aid sampling and replenished as samples were taken. Samples were taken periodically under the reaction conditions over the course of 24 to 48 hours using a double block sampling system.

Liquid samples were analyzed using an Agilent 6890 gas chromatography system equipped with a flame ionization detector, a G133A autosampler, and 30 meter HP-Innowax column (to ensure the separation of para and meta xylenes). The results are shown in FIGS. 13A and 13B. 2,5-Dimethylfuran was observed to inhibit isomerization of para-xylene even when present at very low concentrations relative to the concentration of para-xylene. (See FIGS. 13A and 13B)

### Example 15

### Study of the Effect of the Change in Concentration of 2,5-Hexanedione on the Inhibition of Para-Xylene Isomerization

The inhibition effects of 2,5-hexanedione were tested at various concentrations of 2,5-hexanedione (about 0.2 ml to about 2 ml of 2,5-hexanedione in heptane).

For example, 2 ml of 2,5-hexanedione, 10 ml of para-xylene, 86 ml of n-heptane, and 2 ml of tridecane (as an internal standard) were placed in a reaction vessel. 0.45 g of H—Y zeolite was then added and the flask was purged with N₂ and heated to about 300° C. Once at 300° C., 14 bar (200 psi) of N₂ was added to aid sampling and replenished as samples were taken. Samples were taken periodically under the reaction conditions over the course of 24 to 48 hours using a double block sampling system.

Liquid samples were analyzed using an Agilent 6890 gas chromatography system equipped with a flame ionization detector, a G133A autosampler, and 30 meter HP-Innowax column (to ensure the separation of para and meta xylenes). As can be seen in FIGS. 14A and 14B, para-xylene isomerization was inhibited by the addition of 2,5-hexanedione in heptane solvent. It should be noted that the 2,5-hexanedione underwent cyclization to form 2,5-dimethylfuran upon heating of the reaction vessel. Isomerization inhibition effects could be a result of inhibition by either the diketone, 2,5-dihexanedione, or 2,5-dimethylfuran.

### Example 16

### Study of the Effect of the Ethylene and Water on Para-Xylene Isomerization

The effect of water and ethylene on para-xylene isomerization inhibition was studied under cycloaddition reaction conditions in heptane solvent. Experiments for the effect of water on para-xylene isomerization were conducted by placing 2 ml of water, 10 ml of para-xylene, 86 ml of n-heptane, and 2 ml of tridecane (as an internal standard) in a reaction vessel. 0.45 g H—Y zeolite catalyst was then added and the reaction vessel was purged with N₂ and heated to about 300° C. Once 300° C., 14 bar (200 psi) N₂ was added to aid sampling and replenished as samples were taken. Samples were taken periodically under the reaction conditions over the course of 24 to 48 hours using a double block sampling system. As shown in FIG. 15B, water did not inhibit para-xylene isomerization.

Experiments for the effect of ethylene on para-xylene isomerization were conducted by placing 12 ml of para-xylene, 86 ml of n-heptane, 2 ml of tridecane (as an internal standard) in a reaction vessel. 0.45 g H—Y zeolite catalyst was then added and the reaction vessel was purged with ethylene and heated to about 300° C. Once the reaction temperature was achieved, 14 bar (200 psi) of ethylene gas added to aid sampling and replenished as samples were taken. Samples were taken periodically under the reaction conditions over the course of 24 to 48 hours using a double block sampling system. As shown in FIG. 15A, ethylene was observed to promote the formation of heavy alkylated products in addition to promoting the isomerization of para-xylene.

Liquid samples were analyzed using an Agilent 6890 gas chromatography system equipped with a flame ionization detector, a G133A autosampler, and 30 meter HP-Innowax column (to ensure the separation of para and meta xylenes).

### Example 17

### ²⁷Al MAS NMR Analysis of H—Y Zeolite Catalyst

²⁷Al MAS NMR was used to investigate the aluminum content in H—Y zeolite before and after use in a cycloaddition reaction.²⁷Al MAS NMR spectra were recorded in a fully hydrated state with a Bruker DSX-300 NMR spectrometer at an ²⁷Al frequency of 78.2 MHz, using a 7 mm MAS probe. The spinning rate was 3 kHz and the spectra were acquired with 0.5 s repetition time, 3 μs delay time, and 2000 scans. The chemical shift was referenced to 1.0M aqueous solution of Al(NO₃)₃ (0 ppm). As shown in FIG. 16, the ²⁷Al MAS NMR of spent H—Y zeolite catalyst shows a reduction of tetrahedrally-coordinated aluminum relative to octahedrally-coordinated aluminum when compared to fresh H—Y zeolite catalyst.

### Example 18

### DRIFT Spectroscopy Analysis of H—Y Zeolite Catalyst

FTIR analysis was performed with a Bruker Equinox 55 equipped with a temperature-controlled sample cell and cryogenic MCT detector with 2 cm⁻¹ resolution. A constant helium purge of 20 ml/min flowed through the sample cell to provide an inert atmosphere. A background (KBr) was collected and subtracted from all samples. H—Y zeolite (Zeolyst CBV-600) was degassed at 550° C. for one hour after increasing the temperature from about 120° C. to 550° C. at a ramp rate of 5° C. (min)⁻¹. The degassed zeolite was then saturated with the chemical of interest (2,5-dimethylfuran, p-xylene, or a 1/50^{th} mixture of 2,5-dimethylfuran/p-xylene) at 120° C. Saturation of the catalyst was achieved by redirecting the helium purge stream through a bubble column filled with the chemical of interest. The sample chamber was then heated to about 300° C. and maintained at that temperature for one hour to remove any weakly adsorbed chemicals. The sample was then cooled to 120° C. for spectral acquisition. The DRIFT spectra are shown in FIGS. 17i to 17vii. At 120° C., the spectra showed the disappearance of the Brønsted acid site peaks upon absorption of 2,5-dimethylfuran, para-xylene or mixtures of 2,5-dimethylfuran and para-xylene onto the H—Y zeolite catalyst. (See FIGS. 17ii to 17iv) At 300° C., Brønsted acid sites previously occupied by para-xylene were recovered, while 2,5-dimethylfuran remained absorbed on Brønsted acid sites associated with super- and sodalite cages. (See FIGS. 17v to 17vii)

### Example 19

### Thermo Gravimetric Analysis (TGA) of the Solid Forms

TGA samples were prepared by loading a ceramic cup with the zeolite catalyst, H-beta or H—Y, and heating the sample under a helium atmosphere from a temperature of about 25° C. to about 550° C. at a heating ramp rate of 10° C. (min)⁻¹, and holding at a temperature of 550° C. for one hour to degas the sample. A constant helium purge of 23 ml (min)⁻¹ was passed through the furnace chamber to maintain an inert atmosphere.

After the zeolite catalyst was degassed and cooled to 120° C., 2,5-dimethylfuran or p-xylene was added by redirecting the helium purge stream through a bubble column filled with the chemical of interest. Once the zeolite catalyst was saturated with the chemical of interest, the helium purge stream was removed from the bubble column and redirected back to the furnace chamber to allow for a continuous helium flow. Any physisorbed species were also removed. TGA analysis was then performed under a helium atmosphere at a heating ramp rate of 10° C. (min)⁻¹ with a temperature range of 25° C. to 300° C., with a one hour hold at 300° C. to allow for the removal of adsorbed material. As shown in FIGS. 18A and 18B, incomplete desorption was observed in the TGA analysis of 2,5-dimethylfuran absorbed on H—Y zeolite catalyst. However, complete desorption of para-xylene on the H—Y zeolite catalyst was observed at 300° C. (See FIGS. 18C and 18D)

The relevant teachings of all patents, published applications and literature references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

1. M. J. Climent, A. Corma, S. Iborra, Green Chemistry 2011, 13, 520-540.
2. G. W. Huber, S. Iborra, A. Corma, Chemical Reviews 2006, 106, 4044-4098.
3. C. H. Christensen, J. Rass-Hansen, C. C. Marsden, E. Taarning, K. Egeblad, ChemSusChem 2008,1,283-289.
4. T. R. Carlson, Y.-T. Cheng, J. Jae, G. W. Huber, Energy & Environmental Science 2011, 4, 145-161.
5. Y.-T. Cheng, J. Jae, J. Shi, W. Fan, G. W. Huber, Angewandte Chemie International Edition 2012, 51, 1387-1390.

6. T. Carlson, G. Tompsett, W. Conner, G. Huber, Top Catal 2009, 52, 241-252.
7. A. Corma, S. Iborra, A. Velty, Chem. Rev. (Washington, D.C., U.S.) 2007, 107, 2411-2502.
8. W. Partenheimer, Catalysis Today 1995, 23, 69-158.
9. M. C. Raes, O. J. Proulx, US4506040A, Olin Corp., USA. 1985, p. 9 pp.
10. P. Raghavendrachar, S. Ramachandran, Industrial & Engineering Chemistry Research 1992, 31, 453-462.
11. Gong W H, inventor; 2008 Jun. 10. Terephthalic acid composition and process for the production thereof, U.S. Pat. No. 7,385,081.
12. Brandvold T, inventor; 2012 Nov. 20. Carbohydrate route to para-xylene and terephthalic acid, U.S. Pat. No. 8,314,267.
13. A. M. Gamido Pedrosa, M. J. B. Souza, D. M. A. Melo, A. S. Araujo, Mater. Res. Bull. 2006, 41, 1105-1111.
14. Wilson S. T., Lok, B. M., Flanigen, E. M., inventors, Crystalline Metallophosphate compositions, U.S. Pat. No. 4,310,440
15. Flank, W. H., Wilson S. T., Marta, J. C., Flanigen, E. M., inventors, Process for the production of molecular sieves, U.S. Pat. No. 4,861,743
16. Biaglow, A. I.; Parrillo, D. J.; Gorte, R. J. Journal of Catalysis 1993, 144, 193-201.

What is claimed is:

1. A method for producing para-xylene comprising reacting 2,5-dimethylfuran with ethylene under cycloaddition reaction conditions in the presence of an acidic heterogeneous catalyst and a solvent for 2,5-dimethylfuran to produce para-xylene, wherein the solvent comprises at least 20 wt % of the reaction mixture.

2. The method of claim 1, wherein the acidic heterogeneous catalyst comprises a zeolite molecular sieve, activated carbon, silica, alumina or a non-zeolitic molecular sieve.

3. The method of claim 1, wherein the acidic heterogeneous catalyst comprises a zeolite molecular sieve.

4. The method of claim 3, wherein the zeolite molecular sieve is selected from H—Y, H-ZSM-5 or H-beta.

5. The method of claim 1, wherein the cycloaddition reaction conditions include an elevated temperature.

6. The method of claim 5, wherein the elevated temperature is at least 100° C.

7. The method of claim 5, wherein the elevated temperature is at least 200° C.

8. The method of claim 1, wherein the solvent for 2,5-dimethylfuran is a non-polar solvent.

9. The method of claim 8, wherein the non-polar solvent is a (C5-C20) aliphatic solvent.

10. The method of claim 8, wherein the non-polar solvent is a (C6-C18) aromatic solvent.

11. The method of claim 1, wherein the cycloaddition reaction conditions include an elevated pressure.

12. The method of claim 11, wherein the elevated pressure is at least about 5 bar (73 psi).

13. The method of claim 11, wherein the elevated pressure is at least about 20 bar (290 psi).

14. The method of claim 1, wherein the cycloaddition reaction conditions include a reaction time of at least 1 minute.

15. The method of claim 2, wherein the zeolitic molecular sieve catalyst has a Brønsted acid site density of at least about 0.01 mmol/gram.

16. The method of claim 1, wherein at least 95% of 2,5-dimethylfuran is converted to para-xylene under the reaction conditions.

17. The method of claim 1, wherein the para-xylene is produced in an amount representing at least about 60% yield of para-xylene from the 2,5-dimethylfuran.

18. The method of claim 1, wherein the p-xylene is produced with a selectivity of at least about 60%.

19. The method of claim 1, wherein the acidic heterogeneous catalyst is H—Y and the solvent for 2,5-dimethylfuran is heptane.

20. The method of claim 1, wherein the acidic heterogeneous catalyst is H-beta and the solvent for 2,5-dimethylfuran is heptane.

21. A method for producing para-xylene comprising reacting 2,5-dimethylfuran with ethylene under cycloaddition reaction conditions in the presence of a zeolite molecular sieve catalyst and heptane to produce para-xylene, wherein the heptane comprises at least 20 wt % of the reaction mixture and wherein the cycloaddition reaction conditions include a temperature of at least 250° C., a pressure of at least 20 bar (290 psi), and a reaction time of at least 1 minute.
